(19) United States
(12) Patent Application Publication
(10) Pub. No.: US 2009/0299293 A1
(43) Pub. Date: Dec. 3, 2009

(54) Self-Closing External Vessel Plug With Integrated Wide-Lumen Needle

(75) Inventors: Ernst-Diethelm Harren, Steinhausen (CH); Marc Ulrich Lehmann, Herrliberg (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Inova Medical AG, Cham (CH)

(21) Appl. No.: 12/085,061

(22) PCT Filed: Nov. 9, 2006
(86) PCT No.: PCT/CH2006/000632

§ 371 Date: Mar. 17, 2009

(30) Foreign Application Priority Data
Nov. 15, 2005 (CH) 1832/05

(51) Int. Cl. A61M 5/32

(52) U.S. Cl. 604/177; 604/245

(57) ABSTRACT

A self-closing external vessel plug (1) with an integrated wide-lumen needle for puncturing vessels in the human or animal body has a pressure chamber (8), a closure part (5), a closure element (6), a pressure wall part (7), and flexible wings (13) for fixing on the body. The wide-lumen needle has a cannula (3) integrated with a connection part (2), and a protective cap (12) that can be fitted onto the cannula (3). The cannula (3) penetrates the closure element (6) and the pressure wall part (7). The protective cap (12) that can be fitted onto the cannula (3) is connected to a protective film (11) in such a way that the protective film (11) together with the protective cap (12) is applied to and can be torn off from a layer of medical skin adhesive (10) applied on the pressure wall part (7). The flexible wings (13) are formed by parts of the closure part (5), pressure wall part (7) and protective film (11). An advantage of this wide-lumen needle is that it is much easier to staunch the blood after the puncturing procedure.

The invention relates to a self-closing external vessel plug with an integrated wide-lumen needle for puncturing vessels in the human or animal body, with flexible wings for attachment to the body, according to Patent claim 1.

The invention relates in particular to a further embodiment in conjunction with wide-lumen needles which have a cannula integrated with a connecting part, a flexible connecting hose likewise attached to the connecting part and a protective cap that can be placed on the cannula for the purpose of preventing accidents. The flexible wings, which are usually also secured on the connecting part, serve mainly to facilitate guidance of the cannula in puncturing on the one hand but on the other hand, as already mentioned, serve to allow secure and easy fixation on the human or animal body by means of adhesive tape after puncturing. Exemplary embodiments of objects of this type include, for example, fistula needles.

In the narrower sense, the term “wide-lumen needle” is used as an umbrella term for the parts that belong together in this patent application, namely a wide-lumen cannula, a connecting part and a protective cap for the cannula in the sense of a restriction to what is essential but without the connecting hose (which is usually present).

One example of a conventional fistula needle is model no. 3050 from Suru which is commercially available. It has the design described above and is available in sizes G15, 16 and 17 and also with a connecting hose length of 30 cm; it is evidently available both with and without rotatable flexible wings.

Another example of this type is the fistula needle model no. F16AS from Gambro which is also available commercially. It also has the design described previously and is offered in sizes G14 through G17 and with connecting hose lengths of 15 cm or 30 cm. With this model, the flexible wings are arranged rotatably with respect to the connecting part.

Another example is the so-called “safetouch fistula needle” from Nipro. This needle has a displaceable safety part or sheath part which is in turn equipped with the above-mentioned flexible wings, which are arranged either fixedly or rotatably. The displaceable part serves to protect against accidents.

Alternative approaches which also serve to protect against accidents, but all of which have the aforementioned flexible wings, are disclosed in EP 0 664 139, U.S. Pat. No. 4,941,881 and U.S. Pat. No. 5,120,320, for example.

All these examples have in common the fact that the flexible wings are part of the wide-lumen needle in a defined narrower sense, also they always serve the aforementioned purpose of fixation of the needle by attaching it to the skin with adhesive after puncture and are always also removed again with the withdrawal of the needle. Since the problem of hemostasis occurs immediately after removal of the needle, to which end suitable aids must generally be used and stuck on the skin, it would be helpful at least if adhesives that are already provided would not have to be removed again completely.

The object of the invention is therefore to provide a device for a wide-lumen needle for puncturing vessels in the human or animal body with which the hemostasis to be achieved after a puncture is facilitated.

This object is achieved through the features of Patent claim 1.

This method of achieving the object is based on the fact that elements of a wide-lumen needle of a conventional design can be combined with elements of a self-closing external vessel plug with a pressure chamber, a closure part, a closure element and a pressure wall part as described in Swiss Patent Application No. CH-1731/04.

The inventive self-closing external vessel plug with an integrated wide-lumen needle for puncturing vessels in the human or animal body has flexible wings for securing the needle on the body by means of an adhesive connection. This self-closing external vessel plug has a pressure chamber, a closure part, a closure element and a pressure wall part. The wide-lumen needle has a cannula that is integrated with a connecting part and is provided with an attachable protective cap for protection against accidents, whereby
the cannula passes through the closure element and the pressure wall part,
the protective cap that can be placed on the cannula is connected to a protective film in such a way that the protective film, which can in turn be pulled away with the protective cap, is attached to a medicinal skin adhesive layer attached to the pressure wall part, and
the flexible wings are formed by parts of the closure part, the pressure wall part and the protective film.

A self-closing external vessel plug designed according to the invention with an integrated wide-lumen needle has the advantage that the flexible wings not only facilitate guidance of the needle during insertion and allow secure and easy fixation of the wide-lumen needle on the body after the puncture, but also parts of the device, namely the self-closing external vessel plug with the pressure chamber, the closure part, the closure element and the pressure wall part can remain at the site of the puncture after the cannula has been pulled out and no other separate means need be used to staunch the blood flow.

Another advantage is that the connecting part with the cannula integrated into it or a protective part movably secured in this area need no longer have flexible wings and therefore can be manufactured more easily and less expensively.

Essentially hemostasis with the inventive approach is accomplished by means of endogenous blood because after withdrawing the cannula from the puncture channel, the blood that flows into the pressure chamber creates a backpressure which stops the bleeding. The fact that this is the case is due to the circumstance that the closure element has self-closing properties. However, there are also cases, e.g., in the area of use of fistula needles in which, due to the different shunts installed over a period of time, this active principle can be used in only some patients because the tissue structure between the shunt and the skin does not ensure definitive pressure on the puncture site by means of the patient's own blood in the shunt because of its properties. In such cases, however, there is nevertheless the advantage that the closure element (lenticular shape above) may be used as a pressure aid.

The inventive self-closing external vessel plug with an integrated wide-lumen needle is described in detail below on the basis of an exemplary embodiment with drawings.

In the drawings:

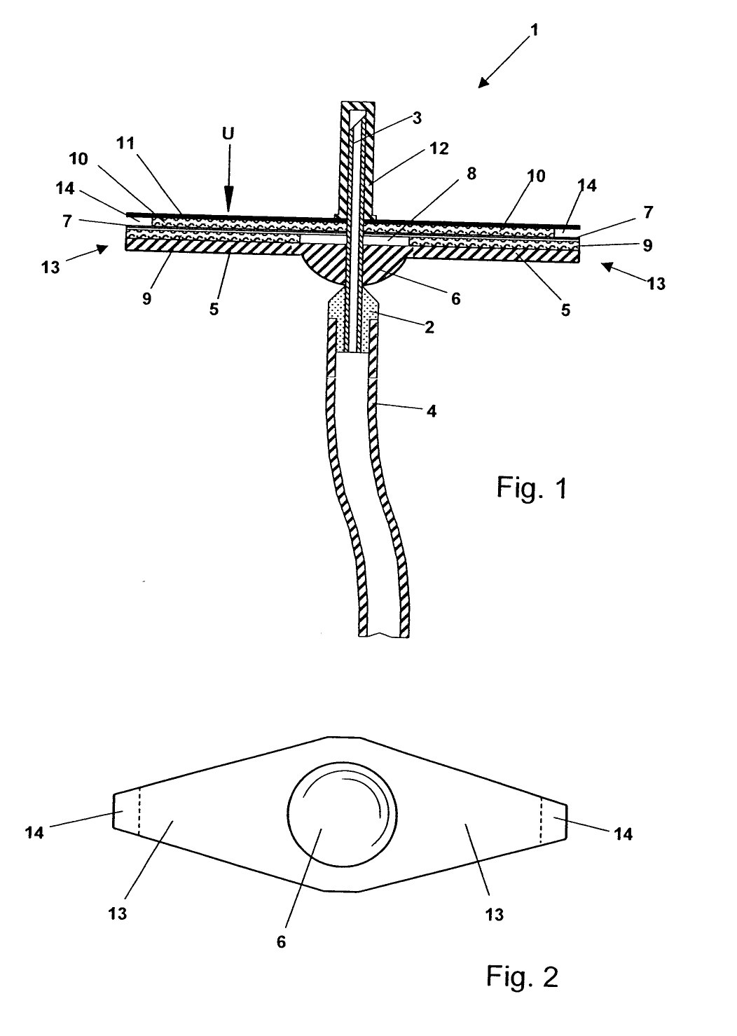

FIG. 1 shows a cross section through a self-closing external vessel plug with an integrated wide-lumen needle (before use), and

FIG. 2 shows a top view of the external vessel plug remaining on the puncture site after withdrawal of the cannula as seen from the side of the closure element.

FIG. 1 shows a cross section through a self-closing external vessel plug 1 with an integrated wide-lumen needle before its use.

The needle has a connecting part 2 with an integrated cannula 3 therein. A flexible connecting hose 4 can be or is attached on the proximal end of the connecting part 2. As is the case with the conventional wide-lumen needles of a similar type, the flexible connecting hose is usually relatively short and is additionally provided with a removable closure and an elastic pressure element that can be pushed onto the hose and is designed to be lockable. However, the latter are not shown in the figures because the connecting hose as well as the accessories mentioned above are not part of the subject matter of the invention.

When placed on the cannula 3 and/or punctured by same, the self-closing external vessel plug 1 has a closure part 5, a closure element 6 integrated into the closure part 5, a pressure wall part 7 and a pressure chamber 8 that is present or can be formed between the closure element 6 and the pressure wall part 7. Both the closure part 5 and the pressure wall part 7 are designed in one piece. The closure element 6 has self-closing properties and is arranged in the part of the pressure chamber 8 facing away from the body and essentially adjacent to the connecting part. The closure element 6 may also be designed in the form of a segment of a sphere or as a lenticular, round or elliptical shape. The closure part 5 and the pressure wall part 7 are joined by means of a technical adhesive layer 9, whereby the adhesive layer 9 is missing in the area of the closure element 6, so that the pressure chamber mentioned above can be formed there. A medicinal skin adhesive layer 10 is provided on the underside U, which is provided for contact with the skin. Furthermore, both the closure part 5 and the pressure wall part 7 as well as the technical adhesive layer 9 and the medicinal skin adhesive layer 10 are preferably transparent.

Due to the design of the self-closing external vessel plug described here, the technical adhesive layer 9 need not have any skin adhesive-specific properties because it cannot even come in contact with the skin.

It is also possible to provide for the adhesive layer 9, which in the simplest case consists only of an adhesive (as mentioned above, also referred to as a technical adhesive, i.e., an adhesive without skin adhesive-specific properties), has a backing film (not shown) which is provided with adhesive on both sides. Such a design of the adhesive layer 9 may be selected to simplify production in the sense that the adhesive layer may also be designed as an “as delivered part” for the production of self-closing external vessel plugs.

The medicinal skin adhesive layer 10, however, is provided for contact with the human or animal skin and may therefore also have antiseptic, anti-allergic or analgesic properties, for example, due to active ingredients that are added.

The medicinal skin adhesive layer 10 is in turn covered with a removable protective film 11, while the partial piece of the cannula 3 protruding out of the self-closing external vessel plug 1 is covered by a protective cap 12, which can also be removed. The protective film 11 and the protective cap 12 are joined together in such a way that the two can be removed jointly from the wide-lumen needle.

Parts of the closure part 5, the pressure wall part 7 and the protective film 11 which form the flexible wings 13 are designed as laterally protruding and opposing extensions. Furthermore, gripping aids 14 are provided on the outer ends of the extensions, which may be areas that are free of skin adhesive, for example.

FIG. 2 shows a top view of the vessel plug 1 remaining after removal of the cannula 3 at the puncture site, as seen from the side of the closure element 6.

The self-closing external vessel plug 1 and the wide-lumen needle are thus integrated into one another according to this invention. To obtain the desired self-closing properties, suitable combinations of materials must be selected. For example, the closure element 6 and the closure part 5 are preferably made of silicone, because with silicone, due to its extremely high restoring force, the puncture channel automatically closes again after the cannula has been withdrawn, so that no blood can flow out. However, the pressure wall part 7 is preferably also made of a soft material which adapts well to the topography of the skin and has a high extensive capacity that is lower than that of the material of the closure element 6. A polyurethane film approximately 25 μm thick is preferably used here, but other materials, such as polyester or polypropylene films, may also be used. The material of the pressure wall part serves, on the one hand, to limit and stabilize the silicone layer in the two-dimensional extent but on the other hand it must also be extensible enough to allow shaping of the pressure chamber when it fills with blood after retracting the cannula 3. Of course, other suitable combinations of materials with which those skilled in the art are familiar may also be taken into consideration.

If a layer structure with a backing film is used for the adhesive layer 9 (as mentioned above) with a (technical) adhesive applied to the backing film on both sides, then of course it is to be expected that this backing film, like the pressure wall part 7 and of course also as a function of the choice of material, will contribute toward limiting the extent and toward stabilization of the closure part 5.

With regard to other details of the self-closing external vessel plug per se, reference is made to Swiss Patent Application CH-1731/04 already cited above.

For use of the inventive self-closing external vessel plug 1 with an integrated wide-lumen needle:
The blood vessel intended for puncturing is located.
The body part to be punctured is cleaned and disinfected in the usual manner.
The protective film 11 attached to the protective cap 3 is pulled away by removing the protective cap 12. The protective film 11 therefore becomes detached from the medicinal skin adhesive layer 10 and the cannula 3 is exposed.
The flexible wings 13 are folded down (in the direction of the connecting hose 4) by means of the gripping aids 14.
The needle (including the connecting hose 4) is aligned for the puncture. This alignment comprises rotation of the needle up to the chamfered tip of the cannula 3 to bring it into the desired orientation.
A puncture is created at the defined location.
The self-closing external vessel plug 1 is adhesively attached to the patient's skin using the medicinal skin adhesive layer 10. No other fastening means are required because the cannula 3 is held in the desired position in a stable manner by the self-closing external vessel plug and in particular by the retaining force of the closure element 6.
After the procedure is finished, the cannula 3 is withdrawn, the protective cap 12 and the protective film 11 are pushed back onto the cannula 3 for safety reasons (risk of injury), but the self-closing external vessel plug 1 remains adhesively applied. By extracting the cannula 3, blood flows out of the puncture channel into the pressure chamber 8, but it cannot escape to the outside because the self-closing properties of the material of the closure element 6 prevent this. However, the pressure chamber 8 expands and fills up with blood until a back pressure has built up by means of the resulting pressure body (the expanding pressure chamber), causing the blood flow to stop. The closure element 6 also ensures that the pressure body is deformed inward, i.e., in the direction of the punctured blood vessel and thereby also achieves the desired effect of staunching the blood flow.

As mentioned above, however, there are cases in which the function principal described above cannot be applied. In such cases, however, the self-closing external vessel plug 1 may be used as a compression aid by applying pressure to the closure element 6 either by the personnel or by the patient himself if he is able to do so.

One area for use of the inventive self-closing external vessel plugs 1 with integrated wide-lumen needles is, in addition to the applications described above, also for fistula needles, for example, such as those used for puncturing inserted shunts on dialysis patients.

Further embodiments of the present invention, e.g., in the field of accident prevention, are of course also possible. For example, the wide-lumen needles could be designed so that a safety sheath could be pushed over the cannula after use in a manner similar to that with the Nipro “safetouch fistula needle” or if a safety sheath were to be automatically pushed over the cannula even by the operation of extracting the cannula out of the closure element.

1. A self-closing external vessel plug (1) with an integrated wide-lumen needle for puncturing vessels in the human or animal body,
characterized in that
the self-closing external vessel plug (1) has a pressure chamber (8), a closure part (5), a closure element (6) and a pressure wall part (7) as well as flexible wings (13) for attachment to the body,
and
the wide-lumen needle has a cannula (3) that is integrated with a connecting part (2) and is provided with an attachable protective cap (12),
whereby
the cannula (3) penetrates through the closure element (6) and the pressure wall part (7),
the protective cap (12) that can be placed on the cannula (3) is connected to a protective film (11) such that the protective film (11) together with the protective cap (12) is detachably applied to a medicinal skin adhesive layer (10), which is in turn applied to the pressure wall part (7), and
the flexible wings (13) are formed by parts of the closure part (5), the pressure wall part (7) and the protective film (11).

2. The self-closing external vessel plug (1) with an integrated wide-lumen needle according to Patent claim 1,
characterized in that
the closure part (5) is designed in one piece and the closure element (6) is integrated therein.

3. The self-closing external vessel plug (1) having an integrated wide-lumen needle according to Patent claim 1,
characterized in that
the pressure wall part (7) is designed in one piece.

4. The self-closing external vessel plug (1) having an integrated wide-lumen needle according to claim 1,
characterized in that
a technical adhesive layer (9) is applied between the closure part (5) and the pressure wall part (7) except for an area beneath the closure element (6), whereby the area that is free of adhesive layer is shaped to form the pressure chamber (8).

5. The self-closing external vessel plug (1) having an integrated wide-lumen needle according to claim 1,
characterized in that
the closure part (5), the pressure wall part (7), a skin adhesive layer (10) and the adhesive layer (9) are preferably transparent.

6. The self-closing external vessel plug (1) having an integrated wide-lumen needle according to claim 1,
characterized in that
the parts of the closure part (5), the pressure wall part (7) and the protective film (11) which form the flexible wings (13) are designed as two laterally protruding and opposite extensions.

7. The self-closing external vessel plug (1) having an integrated wide-lumen needle according to Patent claim 6,
characterized in that
the extensions are provided with gripping aids (14) on the outer ends.

8. The self-closing external vessel plug (1) having an integrated wide-lumen needle according to claim 1,
characterized in that
a connecting hose (4) can be attached to the connecting part (2).

9. The self-closing external vessel plug (1) having an integrated wide-lumen needle according to claim 1,
characterized in that
the closure element (6) is designed in the form of a segment of a sphere or in a lenticular, round or elliptical shape.

10. The self-closing external vessel plug (1) having an integrated wide-lumen needle according to claim 1,
characterized in that
the closure element (6) is arranged in the part of the pressure chamber (8) facing away from the body and essentially adjacent to the connecting part (2) and has self-closing properties.

11. The self-closing external vessel plug (1) having an integrated wide-lumen needle according to Patent claim 10,
characterized in that
the closure element (6) is preferably made of silicone.

12. The self-closing external vessel plug (1) having an integrated wide-lumen needle according to claim 1,
characterized in that
the pressure wall part (7) is made of a soft material that adapts well to the topography of skin and has an extensibility that is high but is lower than that of the material of the closure element (6).

13. The self-closing external vessel plug (1) having an integrated wide-lumen needle according to Patent claim 12,
characterized in that
the pressure wall part (7) is made of a polyurethane, polyester or polypropylene film 5 μm to 50 μm thick, preferably 25 μm thick.

14. The self-closing external vessel plug (1) having an integrated wide-lumen needle according to claim 1,
characterized in that
the adhesive layer (9) need not have any skin adhesive-specific properties.

15. The self-closing external vessel plug (1) having an integrated wide-lumen needle according to claim 1,
characterized in that
the skin adhesive layer (10) has one or more of the properties from the following group of properties due to added active ingredients:
antiseptic properties,
anti-allergic properties,
analgesic properties.
